(19) United States
(12) Patent Application Publication
(10) Pub. No.: US 2010/0202988 A1
(43) Pub. Date: Aug. 12, 2010

(54) CLEAR CARRIER COMPOSITIONS CONTAINING AN ALKOXYLATED MONOACID AND AN ALKYL MONOAMINE AND METHOD OF TREATING KERATINOUS SUBSTRATES USING SUCH COMPOSITIONS

(75) Inventors: Nghi Van Nguyen, Edison, NJ (US); Sawa Hashimoto, Westfield, NJ (US); David W. Cannell, New Hope, NJ (US)

Correspondence Address: L'Oreal USA;Patent Department, 30 L'Oreal Way, Clark, NJ, 07066, US

(73) Assignee: L'OREAL, Paris (FR)

(21) Appl. No.: 12/701,178

(22) Filed: Feb. 5, 2010

Related U.S. Application Data
(60) Provisional application No. 61/150,844, filed on Feb. 9, 2009.
(60) Provisional application No. 61/150,839, filed on Feb. 9, 2009.

(51) Int. Cl. A61K 8/92; A61K 8/41; A61Q 5/12; A61Q 5/10; A61Q 5/08; A61Q 5/02; A61Q 5/00

(52) U.S. Cl. 424/59; 424/62; 424/70.11; 8/405

(57) ABSTRACT

The present invention relates to compositions comprising: (a) at least one alkoxylated monoacid; (b) at least one alkyl monoamine; (c) at least one lipophilic compound; (d) at least one solvent comprising water; and (e) optionally, at least one auxiliary ingredient, wherein the composition is clear in appearance. Such a composition is clear in appearance, and stable. The present invention also relates to a method of making such a clear composition, and to a method of cosmetic treatment of a keratinous substrate using such a composition.

### REFERENCE TO PRIOR APPLICATIONS

This application claims the benefit of priority under 35 U.S.C. §119(e) from U.S. provisional application Ser. Nos. 61/150,844 and 61/150,839 filed Feb. 9, 2009, incorporated herein by reference in their entirety.

### TECHNICAL FIELD

The present invention relates to carrier compositions comprising lipophilic ingredients. More particularly, the present invention provides for a clear composition containing one or more lipophilic ingredients, which remains clear even when diluted in particular with water.

The invention further relates to methods of treating keratinous substrates using such compositions.

### BACKGROUND OF THE DISCLOSURE

Cosmetic and personal care products are available in various forms and one of the forms that are desired by many consumers is a clear aqueous product. At the same time, the consumer expects that such a product will provide desirable cosmetic benefits to keratinous substrates such as hair and skin.

These cosmetic benefits can be provided by the presence of water-insoluble ingredients, for example, oils, silicones and other lipophilic materials, in the product.

However, certain water-insoluble ingredients, which are oftentimes desirable for the treatment of keratinous substrates, are inherently difficult to incorporate into aqueous systems, such as shampoos, conditioners and skin care compositions, without forming a traditional emulsion in either cream or lotion form. Oftentimes, the presence of such ingredients at levels that would impart appreciable cosmetic benefits to hair or skin and/or properties to cosmetic and personal care products result in unstable formulations resulting in undesirable phase separations in aqueous systems.

Therefore, in the formulation of clear aqueous compositions, water-insoluble compounds do not lend themselves to being used therein, due to their inability to significantly associate with the water present in the system. As a result, the presence of these water-insoluble ingredients is generally minimal in personal care products and cosmetic products that employ aqueous systems. Thus, the difficulties in formulating such compositions deprives the consumer of products that can better deliver cosmetic benefits to hair and skin such as conditioning, cleansing, coloring of hair, styling of hair, skin care, and better application and spreadability of products.

Thus, there remains a need for an aqueous composition which can carry increased amounts of water-insoluble materials while remaining both homogeneous and clear in appearance. There also remains a need for an aqueous system which can carry increased amounts of water-insoluble materials such as oils and other lipophilic ingredients in order to deliver desirable benefits to hair and skin.

It has been surprisingly and unexpectedly discovered that the combination of at least one alkoxylated monoacid, at least one alkyl monoamine, at least one solvent, and at least one water-insoluble ingredient, such as a lipophilic compound, yields a composition which is clear in appearance and stable. Moreover, the inventive composition remains clear and stable even if additional solvent such as in particular water is added thereto.

It has also been discovered that the use of this clear composition on keratinous substrates, such as hair and skin, results in desirable and beneficial effects on the substrates, such as in particular improved delivery of active ingredients, improved cosmetic effects such as improved color retention on colored-treated hair, improved conditioning, improved hair styling effects and manageability, improved shine, improved protection from environmental and chemical damage, and enhanced color is the case of coloring compositions

### BRIEF SUMMARY OF THE DISCLOSURE

The present invention is directed to a composition capable of carrying lipophilic ingredients, the composition containing:

(a) at least one alkoxylated monoacid;

(b) at least one alkyl monoamine;

(c) at least one lipophilic compound;

(d) at least one solvent comprising water; and

(e) optionally, at least one auxiliary ingredient.

Such a composition is clear in appearance and stable.

The present invention is also directed to a method of making a clear composition involving the steps of:
(a) providing at least one alkoxylated monoacid;
(b) providing at least one alkyl monoamine;
(c) providing at least one lipophilic compound;
(d) providing at least one solvent comprising water;
(e) optionally, providing at least one auxiliary ingredient; and
(f) mixing the compounds as defined in steps (a) to (e) to form a composition that is clear in appearance.

The present invention is further directed to a method of cosmetic treatment of a keratinous substrate involving the step of applying onto said keratinous substrate a composition as defined herein.

### DETAILED DESCRIPTION OF THE DISCLOSURE

The term “comprising” (and its grammatical variations) as used herein is used in the inclusive sense of “having” or “including” and not in the exclusive sense of “consisting only of”. The terms “a” and “the” as used herein are understood to encompass the plural as well as the singular.

Other than in the operating examples, or where otherwise indicated, all numbers expressing quantities of ingredients and/or reaction conditions are to be understood as being modified in all instances by the term “about”.

The term “lipophilic” means those compounds which are soluble in oils and either completely or partially insoluble in water. In accordance with the present invention, the lipophilic compounds preferably have a solubility in water at 25° C. and at atmospheric pressure of less than 5% by weight, more preferably less than 1% by weight, even more preferably less than 0.5% by weight and better still less than 0.1% by weight.

“At least one” as used herein means one or more and thus includes individual components as well as mixtures/combinations.

The term “clear” as used herein means transparent such that a person is able to see through the composition with their naked eye. The term “clear” as used herein is not meant to encompass those compositions which a person cannot see through with their naked eye such as those which are pearlescent, frosted, hazy, opaque, or cloudy in appearance.

The clarity of the compositions of the present invention can be determined using the McFarland scale, which is based on the McFarland Equivalence Turbidity Standard Test (Remel, 12076 Santa Fe Drive, Lenexa, Kans. 66215, USA). Preferably, the compositions according to the present invention have a McFarland turbidity standard value, as visually determined, equal to or less than 0.5 on the McFarland scale.

The term “stable” as used herein means that the composition does not exhibit phase separation.

The term “carrier system for lipophilic compounds” means a system that delivers a lipophilic ingredient into an aqueous phase by incorporation or solubilization. The lipophilic carrier system of the present invention is capable of bringing lipophilic compounds into an aqueous phase such that the aqueous phase remains clear and stable.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Alkoxylated” as used herein means comprising at least one alkoxy group. As used herein, an alkoxy group is a group corresponding to the formula —O—CHR—(CH₂)ₙ—, wherein R represents H or a C1-C5 alkyl group, and wherein n is an integer ranging from 1 to 6.

Alkoxylated Monoacids

Non-limiting examples of alkoxylated monoacids include compounds corresponding to formula (I):

RO[CH₂O]ᵤ[(CH₂)ₓCH(R′)(CH₂)_{y}(CH₂)_{z}O]ᵥ[CH₂CH₂O]_{w}CH₂COOH (IA)

wherein: R is a hydrocarbon radical containing from about 6 to about 40 carbon atoms;

u, v and w, independently of one another, represent numbers of from 0 to 60;

x, y and z, independently of one another, represent numbers of from 0 to 13;

R′ represents hydrogen, alkyl, and

the sum of x+y+z is ≧0;

Compounds corresponding to formula (IA) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (IA), R is linear or branched, acyclic or cyclic, saturated or unsaturated, aliphatic or aromatic, substituted or unsubstituted. Typically, R is a linear or branched, acyclic C₆₋₄₀ alkyl or alkenyl group or a C₁₋₄₀ alkyl phenyl group, more typically a C₈₋₂₂ alkyl or alkenyl group or a C₄-18 alkyl phenyl group, and even more typically a C₁₂₋₁₈ alkyl group or alkenyl group or a C₆₋₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids of the present invention include, but are not limited to, the following representatives referred to by their INCI names (INCI: nomenclature for raw materials according to the International Cosmetic Ingredient Dictionary, 7^{th} Edition, published by the Cosmetic, Toiletry and Fragrance Association Inc. (CTFA), Washington D.C., USA): Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C₉₋₁₁ Pareth-6 Carboxylic Acid, C₁₁₋₁₅ Pareth-7 Carboxylic Acid, C₁₂₋₁₃ Pareth-5 Carboxylic Acid, C₁₂₋₁₃ Pareth-8 Carboxylic Acid, C₁₂₋₁₃ Pareth-12 Carboxylic Acid, C₁₂₋₁₅ Pareth-7 Carboxylic Acid, C₁₂₋₁₅ Pareth-8 Carboxylic Acid, C₁₄₋₁₅ Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof.

The at least one alkoxylated monoacid is present in the composition in an amount of from about 0.1 to about 50% by weight, such as from about 0.5 to about 30% by weight, and from about 1 to about 20% by weight, based on the weight of the composition as a whole.

Alkyl Monoamines

Non-limiting examples of suitable alkyl monoamines include aliphatic amine compounds corresponding to formula (IA) and their salts:

RN(R′)₂ (IB)

wherein
R is a hydrocarbon radical containing at least 6 carbon atoms. In addition, R is linear or branched, acyclic or cyclic, saturated or unsaturated, aliphatic or aromatic, substituted or unsubstituted. Typically, R is a linear or branched, acyclic alkyl or alkenyl group or an alkyl phenyl group; and R′ is H or a hydrocarbon radical containing at least 6 carbon atoms. In addition, R′ is linear or branched, acyclic or cyclic, saturated or unsaturated, aliphatic or aromatic, substituted or unsubstituted. Typically, R′ is a linear or branched, acyclic alkyl or alkenyl group or an alkyl phenyl group.

Preferred alkyl monoamines include, but are not limited to the following examples: dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine and mixtures thereof.

Other non-limiting examples of alkyl monoamines include amidoamine compounds corresponding to formula (IIB) and its salts:

RCONHR′N(R″)₂ (IIB)

wherein:
R is a hydrocarbon radical containing at least 6 carbon atoms, preferably from 8 to 30 carbon atoms, more preferably from 12 to 24 carbon atoms. In addition, R can be linear or branched, acyclic or cyclic, saturated or unsaturated, aliphatic or aromatic, substituted or unsubstituted. Typically, R is a linear or branched, acyclic alkyl or alkenyl group or an alkyl phenyl group; and
R′ is a divalent hydrocarbon radical containing less than 6 carbon atoms, preferably 2 or 3 carbon atoms, and
R″ is H or a hydrocarbon radical containing less than 6 carbon atoms. In addition, R″ is linear or branched, acyclic or cyclic, saturated or unsaturated, substituted or unsubstituted. Typically, R″ is a linear or branched, acyclic alkyl or alkenyl group. Preferably, R″ is H or a methyl group.

Preferred amidoamines include, but are not limited to the following examples: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, palmitamidopropyl dimethylamine, stearamidoethyldiethylamine, and mixtures thereof.

The at least one alkyl monoamine is present in the composition in an amount of from about 0.1 to about 50% by weight, such as from about 0.5 to about 30% by weight, and from about 1.0 to about 20% by weight, based on the weight of the composition as a whole.

Preferably, the ratio of the acid number of the at least one alkoxylated monoacid to the amine number of the at least one alkyl monoamine is from about 1:10 to about 10:1, and more preferably, is from about 1:5 to about 5:1 and, even more preferably, is from about 1:2 to about 2:1. Also the composition remains clear when diluted with any ratio or amount of additional solvent.

Acid and amine values are generally determined by acid-base titration in the presence of a color indicator based on the European and American Pharmacopoeias and Standard ISO 660.

Lipophilic Compound

The at least one lipophilic compound may, for example, be chosen from oils, fatty esters, hydrocarbon oils, silicones different from alkoxylated silicone acids of the present invention, waxes, fatty acids and salts thereof, fatty alcohols, lipophilic vitamins and esters thereof, organic sunscreens, phospholipids, and mixtures thereof.

Non-limiting examples of oils include plant oil such as olive oil, avocado oil, coconut oil, safflower oil, almond oil, castor oil, jojoba oil, peanut oil, sesame oil, hazelnut oil, sunflower oil, apricot kernel oil, grapeseed oil, linseed oil and palm oil.

Non-limiting examples of hydrocarbon oils include mineral oil, petrolatum, and C₁₀-C₄₀ hydrocarbons which may be aliphatic (with a straight, branched or cyclic chain), aromatic, arylaliphatic such as paraffins, iso-paraffins, isododecanes, aromatic hydrocarbons, and mixtures thereof.

Non-limiting examples of silicones include phenyltrimethicone, dimethicone, cyclomethicone, dimethicone copolyol, laurylmethicone copolyol, cetyl dimethicone, cetyl triethylammonium dimethicone copolyol phthalate, dimethicone copolyol lactate, stearalkonium dimethicone copolyol phthalate, stearaminopropyl dimethicone and polyorganosiloxanes such as polydimethylsiloxane.

Non-limiting examples of waxes include paraffin wax, beeswax, candelilla wax, carnauba wax, jasmine wax, jojoba wax and mimosa wax.

Suitable fatty acids include those containing from 8 to 30, preferably from 12 to 24 carbon atoms, and carboxylate salts of fatty acids. The sodium, potassium, ammonium, calcium and magnesium carboxylates of fatty acids listed are typical examples of the carboxylate salts of the fatty acids.

Non-limiting preferred examples of fatty alcohols include compounds of formula:

R—OH

where R represents a hydrocarbon radical containing at least three carbon atoms, preferably from 8 to 30, more preferably from 12 to 24 carbon atoms, and which may be linear or branched, acyclic or cyclic, saturated or unsaturated, aliphatic or aromatic, substituted or unsubstituted. Typically, R is a linear or branched, acyclic alkyl or alkenyl group or an alkyl phenyl group.

Non-limiting preferred fatty esters include esters formed from fatty acids and C₁₋₁₀ alcohols and esters formed from the fatty alcohols as defined hereabove and C₁₋₄₀ carboxylic acids.

According to a preferred embodiment, the at least one lipophilic compound is chosen from isopropyl palmitate, capric/caprylic triglyceride, isodecyl neopentanoate, polyisobutylene, phloretin, ellagic acid, vitamin D, vitamin E, vitamin E acetate, vitamin A, vitamin A palmitate, 2-oleamido-1,3-octadecanediol, octyl methoxycinnamate, octyl salicylate, 18-methyleicosanoic acid, and mixtures thereof.

The at least one lipophilic compound is present in the composition in a positive amount in an amount of from about 0.1 to about 50% by weight, such as from about 0.1 to about 30% by weight, and from about 0.5 to about 15% by weight, based on the weight of the composition as a whole.

Solvent

The solvent is typically present in an amount from about 10% to about 95% by weight, preferably in an amount from about 50% to about 85% by weight and more preferably from about 60% to 80% by weight, based on the weight of the composition as a whole. The solvent comprises water such as deionized water, alone or in combination with at least one C₁-C₄ alcohol. Alcohols include ethanol, propanol and butanol. Preferably, the alcohol is chosen from ethanol, isopropanol and mixtures thereof.

Auxiliary Ingredients

The composition may optionally contain at least one auxiliary ingredient. The auxiliary ingredient may include film forming agents, proteins, amino acids, cationic conditioners, cationic polymers, nonionic surfactants, anionic surfactants, amphoteric surfactants, zwitterionic surfactants, viscosity modifiers, antibacterial agents, sunscreens, preservatives, pH adjusting agents, bleaching agents, hair dyeing agents, perfumes, sequestering agents, anti-dandruff agents, alpha or beta hydroxy acids or alpha ketoacids, and mixtures thereof.

Non-limiting examples of film forming agents can be chosen from anionic compounds, non-ionic compounds, amphoteric compounds, zwitterionic compounds, proteins, viscosity modifiers, cationic polymers, polyamide, polyaminoamide, polyester, silicone resins, polysaccharides, silicone fluids, polyacrylamide, starches, gums and mixtures thereof.

Non-limiting examples of proteins include collagen, deoxyribonuclease, iodized corn protein, milk protein, protease, serum protein, silk, sweet almond protein, wheat germ protein, wheat protein, alpha and beta helix of keratin proteins, hair proteins, such as intermediate filament proteins, high-sulfur proteins, ultrahigh-sulfur proteins, intermediate filament-associated proteins, high-tyrosine proteins, high-glycine tyrosine proteins, tricohyalin, and mixtures thereof.

Non-limiting examples of amino acids include amino acids derived from the hydrolysis of various proteins as well as the salts, esters, and acyl derivatives thereof. Non-limiting examples of such amino acid agents include amphoteric amino acids such as alkylamido alkylamines, i.e. stearyl acetyl glutamate, capryloyl silk amino acid, capryloyl collagen amino acids, capryloyl keratin amino acids, capryloyl pea amino acids, cocodimonium hydroxypropyl silk amino acids, corn gluten amino acids, cysteine, glutamic acid, glycine, hair keratin amino acids, amino acids such as asparatic acid, threonine, serine, glutamic acid, proline, glycine, alanine, cystine, valine, methionine, isoleucine, leucine, tyrosine, phenylalanine, cysteic acid, lysine, histidine, arginine, cysteine, tryptophan, citrulline, lysine, silk amino acids, wheat amino acids and mixtures thereof.

Non-limiting examples of cationic conditioners include quaternium-27, behenamidopropyl PG-dimonium chloride, hydroxyethyl tallowedimonium chloride, hexadimethrine chloride, stearalkonium chloride and cetrimonium chloride.

Non-limiting examples of cationic polymers include polyquaternium-4, polyquaternium-6, polyquaternium-7, polyquaternium-10, polyquaternium-11, polyquaternium-16, polyquaternium-22 and polyquaternium-32.

Non-limiting examples of nonionic surfactants includes alkoxylated derivatives of the following: fatty alcohols, alkyl phenols, fatty acids, fatty acid esters and fatty acid amides, wherein the alkyl chain is in the C₁₂₋₅₀ range, typically in the C₁₆₋₄₀ range, more typically in the C₂₄ to C₄₀ range, and having from about 1 to about 110 alkoxy groups. The alkoxy groups are selected from the group consisting of C₂-C₆ oxides and their mixtures, with ethylene oxide, propylene oxide, and their mixtures being the typical alkoxides. The alkyl chain may be linear, branched, saturated, or unsaturated. Of these alkoxylated non-ionic surfactants, the alkoxylated alcohols are typical, and the ethoxylated alcohols and propoxylated alcohols are more typical. The alkoxylated alcohols may be used alone or in mixtures with those alkoxylated materials disclosed herein-above.

Representative preferred examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10), steareth-10 (a stearyl alcohol ethoxylate having an average degree of ethoxylation of 10), steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Commercially available corresponding nonionic surfactants are for example Brij® nonionic surfactants from Croda, Inc., Edison, N.J. Typically, Brij® is the condensation products of aliphatic alcohols with from 1 to 54 moles of ethylene oxide, the alkyl chain of the alcohol being typically a linear chain and having from 8 to 22 carbon atoms, for example, Brij 72 (i.e., Steareth-2) and Brij 76 (i.e., Steareth-10).

Also useful herein as nonionic surfactants are alkyl glycosides, which are the condensation products of long chain alcohols, which are the condensation products of long chain alcohols, e.g. C₈-C₃₀ alcohols, with sugar or starch polymers. These compounds can be represented by the formula (S)n —O—R wherein S is a sugar moiety such as glucose, fructose, mannose, galactose, and the like; n is an integer of from about 1 to about 1000, and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C₈-C₂₀ alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein sucrose ester surfactants such as sucrose cocoate and sucrose laurate.

Other nonionic surfactants suitable for use in the present invention are glyceryl esters and polyglyceryl esters, including but not limited to, glyceryl monoesters, typically glyceryl monoesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C₁₆-C₂₂ saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Croda, Inc., Edison, N.J.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use as nonionic surfactants are alkoxylated derivatives of glyceryl esters, sorbitan esters, and alkyl polyglycosides, wherein the alkoxy groups is selected from the group consisting of C₂-C₆ oxides and their mixtures, with ethoxylated or propoxylated derivatives of these materials being typical. Nonlimiting examples of commercially available ethoxylated materials include ethoxylated sorbitan mono-, di- and/or tri-esters of C₁₂ to C₁₈ fatty acids with an average degree of ethoxylation of from 2 to 20, such as the products sold under the name TWEEN® by the company Uniqema).

Non-limiting examples of anionic surfactants include compounds in the classes known as alkyl sulfates, alkyl ether sulfates, alkyl sulfonates, alkyl ether sulfonates, sulfate esters of an alkylphenoxy polyoxyethylene ethanol, alpha-olefin sulfonates, beta alkyloxy alkene sulfonates, alkyl arylsulfonates, alkyl carbonates, alkyl ether carboxylates, fatty acids, succinamates, sulfosuccinates, sarcosinates, octoxynol or nonoxynol phosphates, taurates, fatty taurides, sulfated monoglycerides, fatty acid amino polyoxyethylene sulfates, isethionates and mixtures thereof. Specific examples of anionic surfactants include the ammonium, monoethanolamine, diethanolamine, triethanolamine, isopropylamine, sodium, potassium, lithium, or magnesium salts of lauryl sulfate, dodecylbenzene-sulfonate, lauryl sulfosuccinate, lauryl ether sulfate, lauryl ether carboxylate, lauryl sarcosinate, cocomethyl tauride, and sulfosuccinate half ester amide and mixtures thereof.

Non-limiting examples of amphoteric and zwitterionic surfactants include alkyl, alkyl dimethyl, alkylamido, alkyl amide, alkylamidopropyl, or alkyl dimethylammonium betaine; alky amidopropyl or alkyl sulfobetaine; alkyl, alkylampho, or alkylamphocarboxy glycinate; alkyl, or alkyl substituted imidazoline mono or dicarboxylate; sodium salts of alkyl mono- or dicarboxylates; alkyl beta amino acids; alkyl amidopropyl, or alkyl ether hydroxysultaine; alkyl amidopropyl dimethyl ammonia acetate; alkyl ampho mono- or diacetate; alkyl, or alkyl ampho, or alkyl imino dipropionate; alkyl amphopropionate; alkyl beta amino propionic acid; alkyl dipropionate; alkyl beta iminodipropionate; branched or n-alkyl dimethylamidopropionate; alkyl carboxylated propionate; alkyl, or methyl alkyl imidazoline; fluorinated alkyl amphoteric mixtures.

Specific examples include cocamidopropyl betaine, lauramidopropyl betaine, coco/oleamidopropyl betaine, coco betaine, oleyl betaine, cocamidopropyl hydroxysultaine, tallowamidopropyl hydroxysultaine and dihydroxyethyl tallow glycinate and mixtures thereof.

Non-limiting examples of viscosity modifiers include water swellable/soluble cationic polymers from quaternized polysaccharides such as trimethyl ammonium substituted epoxide of hydroxyethyl cellulose, diallyldimethyl ammonium salts of hydroxyethylcellulose, deacylated chitin or chitosan, dihydroxypropyl chitosan trimonium chloride, hydroxypropltrimethyl ammonium chloride guar, locust bean, or konjac mannan gum; quaternized synthetics such as acrylamide dimethyl diallyl ammonium chloride copolymers, acrylamide/dimethyl diallyl ammonium chloride/acrylic acid terpolymer, quaternized poly (vinyl pyrrolidone/dimethyl amino ethylmethacrylate), poly (vinylpyrrolidone/methacrylamidopropyl trimethylammonium chloride), polyvinyl pyrrolidone/methylvinylimidazolinium chloride or methyl sulfate copolymer, chloroethylether/dimethylaminopropylamine/adipate or azelate terpolymer, vinylpyrrolidone/methacrylamidopropyl trimethylammonium chloride, acrylonitrile/acrylic acid/dimethylpropanediammonium acrylates sulfate terpolymer. Anionic or nonionic polysaccharide polymers such as gum tragacanth, sodium or propylene glycol alginate, kappa-, iota-, or lambda-carrageenan, guar or hydroxylpropyl guar gum, karaya gum, gum Arabic, locust bean gum, konjac mannan gum, gellan, xanthan, succinoglycan or its acidic or enzymatic hydrolysates, sodium carboxymethyl cellulose, methycellulose, hydroxylethylcellulose, hydroxypropylmethylcellulose, and hydroxypropylecellulose; and/or hydrophobically modified anionic, cationic, or nonionic polymers such as, but not limited to, alkyl and/or substituted hydroxyethylcellulose, lauryl dimethyl ammonium substituted epoxide of hydroxyethylcellulose, propoxylated cellulosic, xanthan, succinoglycan, or polygalactomannoses, alkyl methacrylates/crosslinked acrylic acid copolymer and/or acrylonitrile/acrylates block copolymer.

Non-limiting examples of antibacterial agents include bacitracin, phenol, benzethonium chloride, erythromycin, neomycin, tetracycline, chlortetracycline and mixtures thereof.

Non-limiting examples of sunscreens include benzophenones, bornelone, butyl PABA, cinnamidopropyl trimethyl ammonium chloride, disodium distryrylbiphenyl disulfonate, PABA, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, ethylhexyl dimethyl PABA, red petrolatum, and mixtures thereof.

Non-limiting examples of preservatives include ethanol, polyvinyl alcohol, phenoxyethanol, benzyl alcohol, methyl paraben, propyl paraben and mixtures thereof.

Non-limiting examples of pH adjusting agents include potassium acetate, sodium carbonate, sodium hydroxide, phosphoric acid, succinic acid, sodium citrate, citric acid, boric acid, lactic acid, sodium hydrogen carbonate and mixtures thereof.

Bleaching agents include, but not limited to, hydrogen peroxide, perborate and persufate salts. EDTA and other aminocarboxylates may be used as sequestering agents. Anti-dandruff agents such as zinc pyrithione, salicylic acid, climbazole, ketoconazole, sulfur piroctone olamine, selenium sulfide and mixtures thereof may also be used as an auxiliary ingredient.

The alpha hydroxy acids may exist in the keto acid form, or the ester form. Examples of such alpha hydroxy acids include glycolic acid, malic acid, pyruvic acid, mandelic acid, lactic acid, methyllactic acid, and mixtures thereof.

Also beta hydroxy acids such as salicylic acid, and derivatives thereof may be included in the compositions of the present invention. In addition, mixtures of the above alpha and beta hydroxyl acids or alpha ketoacids can be advantageously included.

The compositions described above are useful in compositions for treating keratinous substrates. Theses compositions include hair care products such as shampoos and conditioners, products for treating skin such as skin cleansers and personal hygiene products and products for cleaning and treating lips and nails.

For example, when the keratinous substrate being treated is hair, the compositions of the invention may impart shine, conditioning, color retention in particular when the compositions are formulated into a rinse-off product. In this case, the method of the present invention will include a rinsing step usually performed with water, after a leave-on time of the composition of at least 30 seconds.

Similar properties, along with styling, may be provided when the composition is in the form of a leave-on product.

When the keratinous substrate is skin, the compositions may impart protection from the sun (sunscreens) or provide skin benefits by serving as a carrier vehicle for skin actives (anti-acne, anti-wrinkle, etc.).

The method of treatment to be provided will depend on the keratinous substrate being targeted and, consequently, the specific ingredients contained in the composition used to effectuate the treatment. One of ordinary skill in the art will easily be able to determine these variables. Regardless of the type of treatment and/or the type of keratinous substrate chosen, the method of treatment will be performed by a composition which is clear in appearance, regardless of the degree of dilution.

### EXAMPLE

The following examples are for illustrative purposes only and are not intended to limit the scope of the claims.

### Example 1

Various compositions (all of them qs to 100% with water), each containing a lipophilic compound, were prepared using the ingredients listed in Table 1 below, in which all quantities as expressed as percentages by weight with regard to the total weight of the composition.

These compositions were characterized as either clear or opaque using the McFarland scale. The McFarland scale is based on the McFarland Equivalence Turbidity Standard Test (Remel, 12076 Santa Fe Drive, Lenexa, Kans. 66215, USA). McFarland standards are used most commonly in microbiology as a reference to measure turbidity of bacterial suspensions in test tubes. The standards are generally prepared suspensions of either barium chloride or latex that range from a scale of 0.5 to 4. The higher the number, the more turbid the suspension. The latex suspension standard was used in this study.

Each composition in this study was placed into a clear glass test tube and was visually compared to the McFarland standards against a white card with contrasting black lines. A composition that did not exhibit phase separation, but visually appeared to possess a McFarland turbidity standard value of greater than 0.5 (>0.5) on the McFarland scale was deemed to be opaque. Conversely, a composition that did not exhibit phase separation, but visually appeared to possess a McFarland turbidity standard value equal to, or less than 0.5 (≦0.5) on the McFarland scale was deemed to be clear.

| | Alkoxylated | | Lipophilic | |
|---|---|---|---|---|
| | monoacid | Alkyl monoamine | compound | Properties |
| | Laureth-11 | Oleamidopropyl | Retinyl | ≦0.5(clear + |
| | Carboxylic Acid, | Dimethylamine, 4% | Palmitate, 1% | homogeneous) |
| | 4.5% | | | |
| 1b. | Laureth-11 | — | Retinyl | >0.5(opaque) |
| | Carboxylic Acid, | | Palmitate, 1% | |
| | 4.5% | | | |
| 1c. | — | Oleamidopropyl | Retinyl | Separated |
| | | Dimethylamine, 4% | Palmitate, 1% | |
| 2a. | Laureth-11 | Behenamidopropyl | Isodecyl | ≦0.5(clear + |
| | Carboxylic Acid, | Dimethylamine, 2% | Neopentanoate, | homogeneous) |
| | 4.5% | | 1% | |
| 2b. | Laureth-11 | — | Isodecyl | >0.5(opaque) |
| | Carboxylic Acid, | | Neopentanoate, | |
| | 4.5% | | 1% | |
| 2c. | — | Behenamidopropyl | Isodecyl | Separated |
| | | Dimethylamine, 2% | Neopentanoate, | |
| | | | 1% | |
| 3a. | Laureth-11 | Stearamidopropyl | Isodecyl | ≦0.5(clear + |
| | Carboxylic Acid, | Dimethylamine, 2% | Neopentanoate, | homogeneous) |
| | 4.5% | | 1% | |
| 3b. | Laureth-11 | — | Isodecyl | >0.5(opaque) |
| | Carboxylic Acid, | | Neopentanoate, | |
| | 4.5% | | 1% | |
| 3c. | — | Stearamidopropyl | Isodecyl | Separated |
| | | Dimethylamine, 2% | Neopentanoate, | |
| | | | 1% | |
| 4a. | Laureth-11 | Brassicaainidopropyl | Isodecyl | ≦0.5(clear + |
| | Carboxylic Acid, | Dimethylamine, 2% | Neopentanoate, | homogeneous) |
| | 4.5% | | 1% | |
| 4b. | Laureth-11 | — | Isodecyl | >0.5(opaque) |
| | Carboxylic Acid, | | Neopentanoate, | |
| | 4.5% | | 1% | |
| 4c. | — | Brassicaamidopropyl | Isodecyl | Separated |
| | | Dimethylamine, 2% | Neopentanoate, | |
| | | | 1% | |

The results above show that the compositions having either the alkoxylated monoacid or the alkyl monoamine exhibited phase separations or appeared cloudy (>0.5 rating on the McFarland scale). In contrast, the compositions having both the alkoxylated monoacid and the alkyl monoamine did not exhibit phase separations and were clear (≦0.5 rating on the McFarland scale).

### Example 2

Efficiency of the Presently Claimed Composition

Four (4) 3 g normal virgin hair (chemically untreated hair) swatches were treated with 100 g of the following solutions for 3 hrs, then rinsed for 1 minute.

### Treatment 1: Laureth-11 Carboxylic Acid 4.5%; Oleamidopropyl Dimethylamine 4%; Vitamin A (Retinyl Palmitate) 1%; Water Q.S. to 100% (Clear)

### Treatment 2: Laureth-11 Carboxylic Acid 4.5%; Vitamin A (Retinyl Palmitate) 1%; Water Q.S. to 100% (Hazy)

### Treatment 3: Oleamidopropyl Dimethylamine 4%; Vitamin A (Retinyl Palmitate) 1%; Water, Q.S. to 100% (Separated)

### Treatment 4: Vitamin A (Retinyl Palmitate) 1%; Water Q.S. to 100% (Separated)

Swatches were dried completely, then 1 g of the hair was placed into 15 g of ethanol overnight with gentle shaking. The amount of Vitamin A extracted from the hair was determined by measuring the UV-V is absorbance of the ethanol solution at 325 nm. The results are shown below:

| Treatment | mg of Vit A/g hair |
|---|---|
| 1 | 0.34 |
| 2 | 0.12 |
| 3 | 0.00 |
| 4 | 0.00 |

The data indicate that hair treated with Treatment 1 showed the highest amount of Vitamin A which is statistically significant from all other treatments. Therefore, the presently claimed composition delivers Vitamin A to hair significantly better than any other compositions lacking one or more of its components.

### Example 3

Three (3) normal virgin hair (chemically untreated hair) swatches were prepared for wet combing measurements by shampooing each swatch twice with a commercial product, Redken Hair Cleansing Crème, for 1 minute and rinsing each swatch under water for 1 minute. Initial wet combing measurements were taken using the Instron Tensile Tester Model 4444. The swatches were then treated with 0.5 g of solution/g hair of the treatment below for 1 minute, and rinsed under water for 10 seconds (80 gallons per hour (gph), 32C). Final wet combing measurements were then taken.

Treatment: Laureth-11 Carboxylic Acid 4.5%, Oleamidopropyl Dimethylamine 2%, Isodecyl Neopentanoate 1%, Water Q.S. to 100% (Clear)

Wet Combing Results

| Initial energy to comb the | Final energy to comb the |
|---|---|
| hair swatch (g*in) | hair swatch (g*in) |
| 392.75 | 140.98 |

The wet combing results showed that the amount of energy required to comb the hair after treating the hair with the inventive composition was statistically significant less than the amount of energy required to comb the hair before treating the hair with the inventive composition. Less energy required to comb wet hair means that it is easier to comb the hair. Therefore, the inventive composition imparted a better and more desirable wet combing property to hair.

The foregoing description illustrates and describes the present disclosure. Additionally, the disclosure shows and describes only the preferred embodiments of the disclosure, but, as mentioned above, it is to be understood that it is capable of changes or modifications within the scope of the concept as expressed herein, commensurate with the above teachings and/or skill or knowledge of the relevant art. The embodiments described hereinabove are further intended to explain best modes known of practicing the invention and to enable others skilled in the art to utilize the disclosure in such, or other, embodiments and with the various modification required by the particular applications or uses disclosed herein. Accordingly, the description is not intended to limit the invention to the form disclosed herein. Also, it is intended that the appended claims be construed to include alternative embodiments.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

What is claimed is:

1. A composition comprising:
(a) at least one alkoxylated monoacid;
(b) at least one alkyl monoamine;
(c) at least one lipophilic compound;
(d) at least one solvent comprising water; and
(e) optionally, at least one auxiliary ingredient.

2. The composition of claim 1 wherein the at least one alkoxylated monoacid is chosen from Laureth-5 Carboxylic Acid, Laureth-11 Carboxylic Acid, C₁₂₋₁₃ Pareth-5 Carboxylic Acid, C₁₂₋₁₃ Pareth-8 Carboxylic Acid Myreth-3 Carboxylic Acid, Myreth-5 Carboxylic Acid, Nonoxynol-5 Carboxylic Acid, Nonoxynol-10 Carboxylic Acid, Oleth-3 Carboxylic Acid, Oleth-Carboxylic Acid, Ceteth-13 Carboxylic Acid, Deceth-2 Carboxylic Acid, Isosteareth-11 Carboxylic Acid, Trideceth-7 Carboxylic Acid, Trideceth-15 Carboxylic Acid, Undeceth-5 Carboxylic Acid, and mixtures thereof.

3. The composition of claim 1 wherein the at least one alkoxylated monoacid is present in an amount of from about 0.1 to about 50% by weight.

4. The composition of claim 1 wherein the at least one alkoxylated monoacid is present in an amount of from about 0.5 to about 30% by weight.

5. The composition of claim 1 wherein the at least one alkoxylated monoacid is present in an amount of from about 1.0 to about 20% by weight.

6. The composition of claim 1 wherein the at least one alkyl monoamine is chosen from
aliphatic amine compounds corresponding to formula (IB) and their salts:
RN(R′)₂ (IB)
wherein
R is a hydrocarbon radical containing at least 6 carbon atoms;
R′ is H or a hydrocarbon radical containing less than 6 carbon atoms;
amidoamine compounds corresponding to formula (IIB) and their salts:
RCONHR′N(R″)₂ (IIB)
wherein:
R is a hydrocarbon radical containing at least 6 carbon atoms;
R′ is a divalent hydrocarbon radical containing less than 6 carbon atoms; and
R″ is H or a hydrocarbon radical containing less than 6 carbon atoms, and mixtures thereof.

7. The composition of claim 1 wherein the at least one alkyl monoamine is chosen from dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine and mixtures thereof.

8. The composition of claim 1 wherein the at least one alkyl monoamine is chosen from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, palmitamidopropyl dimethylamine, stearamidoethyldiethylamine, and mixtures thereof.

9. The composition of claim 1 wherein the at least one alkyl monoamine is present in an amount of from about 0.1 to about 50% by weight.

10. The composition of claim 1, wherein the at least one alkyl monoamine is present in an amount ranging from 0.5 to 30% by weight, based on the total weight of the composition.

11. The composition of claim 1 wherein the at least one alkyl monoamine is present in an amount of from about 1.0 to about 20% by weight.

12. The composition of claim 1 wherein the ratio of the acid number of the at least one alkoxylated monoacid to the amine number of the at least one alkyl monoamine is from about 1:10 to about 10:1.

13. The composition of claim 1 wherein the ratio of the acid number of the at least one alkoxylated monoacid to the amine number of the at least one alkyl monoamine is from about 1:5 to about 5:1.

14. The composition of claim 1 wherein the ratio of the acid number of the at least one alkoxylated monoacid to the amine number of the at least one alkyl monoamine is from about 1:2 to about 2:1.

15. The composition of claim 1, wherein the at least one lipophilic compound is chosen from oils, fatty esters, hydrocarbon oils, silicones different from said alkoxylated silicone acids, waxes, fatty acids and salts thereof, fatty alcohols, lipophilic vitamins and esters thereof, organic sunscreens, phospholipids, and mixtures thereof.

16. The composition of claim 1, wherein the at least one lipophilic compound is present in an amount of from 0.1 to 50% by weight, based on the total weight of the composition.

17. The composition of claim 1, wherein the at least one lipophilic compound is present in an amount of from 0.1 to 30% by weight, based on the total weight of the composition.

18. The composition of claim 1, wherein the at least one lipophilic compound is present in an amount of from 0.5 to 15% by weight, based on the total weight of the composition.

19. The composition of claim 1, wherein the solvent is present in an amount from 10 to 95% by weight, based on the total weight of the composition.

20. The composition of claim 1, wherein the solvent is present in an amount from 50 to 85% by weight, based on the total weight of the composition.

21. The composition of claim 1, wherein the solvent is present in an amount from 60 to 80% by weight, based on the total weight of the composition.

22. The composition of claim 1, wherein the solvent comprises water, alone or in combination with at least one C₁-C₄ alcohol.

23. The composition claim 1, wherein the solvent comprises at least 20% by weight of water.

24. The composition claim 1, wherein the solvent comprises at least 50% by weight of water.

25. The composition claim 1, wherein the solvent comprises at least 80% by weight of water.

26. The composition claim 1, wherein the at least one auxiliary ingredient is chosen from film forming agents, amino acids, proteins, cationic conditioners, cationic polymers, anionic surfactants, nonionic surfactants, amphoteric surfactants, zwitterionic surfactants, viscosity modifiers, antibacterial agents, sunscreens, preservatives, pH adjusting agents, bleaching agents, hair dyeing agents, perfumes, sequestering agents, anti-dandruff agents and mixtures thereof.

27. A method of making a clear composition involving the steps of:
(a) providing at least one alkoxylated monoacid;
(b) providing at least one alkyl monoamines;
(c) providing at least one lipophilic compound;
(d) providing at least one solvent comprising water;
(e) optionally, providing at least one auxiliary ingredient; and
(f) mixing the compounds as defined in steps (a) to (e) to form a composition that is clear in appearance.

28. A method of treating a keratinous substrate comprising applying onto the keratinous substrate a composition containing:
(a) at least one alkoxylated monoacid;
(b) at least one alkyl monoamine;
(c) at least one lipophilic compound;
(d) at least one solvent comprising water; and
(e) optionally, at least one auxiliary ingredient.

29. The method of claim 28 wherein the at least one alkoxylated monoacid is chosen from Laureth-5 Carboxylic Acid, Laureth-11 Carboxylic Acid, C₁₂₋₁₃ Pareth-5 Carboxylic Acid, C₁₂₋₁₃ Pareth-8 Carboxylic Acid Myreth-3 Carboxylic Acid, Myreth-5 Carboxylic Acid, Nonoxynol-5 Carboxylic Acid, Nonoxynol-10 Carboxylic Acid, Oleth-3 Carboxylic Acid, Oleth-Carboxylic Acid, Ceteth-13 Carboxylic Acid, Deceth-2 Carboxylic Acid, Isosteareth-11 Carboxylic Acid, Trideceth-7 Carboxylic Acid, Trideceth-15 Carboxylic Acid, Undeceth-5 Carboxylic Acid, and mixtures thereof.

30. The method of claim 28 wherein the at least one alkyl monoamine is chosen from
aliphatic amine compounds corresponding to formula (IB) and their salts:
RN(R′)₂ (IB)
wherein
R′ is a hydrocarbon radical containing at least 6 carbon atoms;
R′ is H or a hydrocarbon radical containing less than 6 carbon atoms;
amidoamine compounds corresponding to formula (IIB) and their salts:
RCONHR′N(R″)₂ (IIB)
wherein:
R is a hydrocarbon radical containing at least 6 carbon atoms;
R′ is a divalent hydrocarbon radical containing less than 6 carbon atoms; and
R″ is H or a hydrocarbon radical containing less than 6 carbon atoms, and mixtures thereof.

31. The method of claim 28 wherein the at least one alkyl monoamine is chosen from dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine and mixtures thereof.

32. The method of claim 28 wherein the at least one alkyl monoamine is chosen from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, palmitamidopropyl dimethylamine, stearamidoethyldiethylamine, and mixtures thereof.

33. The method of claim 28 wherein the ratio of the acid number of the at least one alkoxylated monoacid to the amine number of the at least one alkyl monoamine is from about 1:10 to about 10:1.

34. The method of claim 28 wherein the ratio of the acid number of the at least one alkoxylated monoacid to the amine number of the at least one alkyl monoamine) is from about 1:5 to about 5:1.

35. The method of claim 28 wherein the ratio of the acid number of the at least one alkoxylated monoacid to the amine number of the at least one alkyl monoamine is from about 1:2 to about 2:1.

36. The method of claim 28, wherein the at least one lipophilic compound is chosen from oils, fatty esters, hydrocarbon oils, silicones different from said alkoxylated silicone acids, waxes, fatty acids and salts thereof, fatty alcohols, lipophilic vitamins and esters thereof, organic sunscreens, phospholipids, and mixtures thereof.

37. The method of claim 28, wherein the solvent is present in an amount from 10 to 95% by weight, based on the total weight of the composition.

38. The method of claim 28, wherein the solvent comprises water, alone or in combination with at least one C₁-C₄ alcohol.

39. The method of claim 28 wherein the at least one auxiliary ingredient is chosen from film forming agents, amino acids, proteins, cationic conditioners, cationic polymers, anionic surfactants, nonionic surfactants, amphoteric surfactants, zwitterionic surfactants, viscosity modifiers, antibacterial agents, sunscreens, preservatives, pH adjusting agents, bleaching agents, hair dyeing agents, perfumes, sequestering agents, anti-dandruff agents and mixtures thereof.
